(19) United States
(12) Patent Application Publication
(10) Pub. No.: US 2007/0003639 A1
(43) Pub. Date: Jan. 4, 2007

(54) Preparation for oral administration containing physiologically active fatty acids and oligomer proanthocyanidin

(76) Inventors: Catherine Le Hen Ferrenbach, Meaux (FR); Bernd Fabry, Korschenbroich (DE); Santiago Rull Prous, Barcelona (ES); Christophe Carite, Rilhac-Rancon (FR); Sybille Buchwald-Werner, Dusseldorf (DE)

Correspondence Address: COGNIS CORPORATION;PATENT DEPARTMENT, 300 BROOKSIDE AVENUE, AMBLER, PA, 19002, US

(21) Appl. No.: 10/553,374

(22) PCT Filed: Apr. 3, 2004
(86) PCT No.: PCT/EP04/03558

§ 371 Date: Aug. 21, 2006

(30) Foreign Application Priority Data
Apr. 14, 2003 (DE) 103171096

(51) Int. Cl. A61K 36/87; A61K 36/82; A61K 31/7048; A61K 31/353

(52) U.S. Cl. 424/729; 424/766; 424/769; 514/27; 514/456

(57) ABSTRACT

Compositions for oral administration of (a) a physiologically active fatty acid containing about 16 to 26 carbon atoms and about 2 to 6 double bonds, and esters or glycerides thereof; and (b) an oligomeric proanthocyanidin or a plant extract containing said proanthocyanidin are disclosed. Methods of using the compositions as additives in food products are also disclosed.

### FIELD OF THE INVENTION

This invention relates generally to food additives or supplements and, more particularly, to new preparations for oral application containing special unsaturated fatty acids together with special polyphenols.

### PRIOR ART

In recent years, the market for food additives has experienced a huge upswing. There is a demand among consumers both for products which utilize physical wellbeing and increase the body's defences in a rather undifferentiated manner, as is typical of vitamins for example, and for products known as health foods or dietary supplements which, for example, accelerate fat degradation or muscle buildup. According to International patent application WO 97/46230 (WARF), for example, conjugated linoleic acid may be used for this purpose. Another example of the growing market for dietary supplements may be placed under the heading of “cosmetic inside” or “beauty inside”. Here, the objective is to support the skin, hair and finger nails in their physiological function and to slow down such phenomena as ageing of the skin for example. Carotinoids, for example, have long been known for such applications, affording protection against the sun.

The problem addressed by the present invention, for example, was both to increase the known lipogenase-inhibiting properties of substances, such as conjugated linoleic acid, for example, or derivatives thereof, and to add a new quality to the performance profile, namely to regulate the moisture content of the skin.

### DESCRIPTION OF THE INVENTION

The present invention relates to preparations for oral application containing
(a) physiologically active fatty acids containing 16 to 26 carbon atoms and 2 to 6 double bonds, esters or glycerides thereof and
(b) oligomeric proanthocyanolidins (OPCs) or plant extracts containing them.

It has surprisingly been found that, when taken orally, the combination of the long-chain unsaturated fatty acids and the special polyphenols leads to a synergistically improved lipogenase inhibition and increases the liquid drainage. These effects can be utilized both to support the degradation of body fats, for example in a diet, and to regulate the moisture content of the skin and, in doing so, effectively to combat the symptoms of dry skin.

Physiologically Active Fatty Acids

A common criterion of the physiologically active fatty acids used as component (a) is that they have a sufficiently long lipid component and a sufficient number of double bonds. Accordingly, fatty acids containing 18 to 24 carbon atoms and 2 to 5 double bonds are suitable for this purpose.

In a first embodiment of the invention, conjugated linoleic acid (CLA), esters thereof—more especially those with lower aliphatic alcohols containing 1 to 4 carbon atoms—or glycerides thereof—more especially the synthetic triglycerides—are used for this purpose. These are all known substances which are normally produced by base-catalyzed isomerization of thistle oil or corresponding alkyl esters and subsequent enzymatic hydrolysis. It has proved to be of advantage for the CLA or CLA derivatives to meet a certain specification, according to which the acyl component contains at least 30% by weight t10,c12 isomers, at least 30% by weight c9,t11 isomers and, in all, less than 1% by weight 8,10-, 11,13- and t,t-isomers. Corresponding products are marketed, for example, under the name of Tonalin® CLA-80 (Cognis).

In a second alternative embodiment, component (a) may also be formed by so-called omega-3 fatty acids which typically contain 18 to 26 and more particularly 20 to 22 carbon atoms and at least 4 to 6 double bonds. These substances are also obtainable by standard methods of organic chemistry, for example by transesterification of fish oil, urea precipitation of the alkyl esters obtained and subsequent extraction with nonpolar solvents, as described in German patent DE 3926658 C2 (Norsk Hydro). Fatty acid mixtures rich in omega-3 (all-Z)-5,8,11,14,17-eicosapentaenoic acid (EPA) C 20:5 and (all-Z)-4,7,10,13,16,19-docosahexaenoic acid (DHA) C 22:6 are obtained in this way. Such products are marketed, for example, under the name of Omacor® (Pronova).

Oligomeric Procyanolidins

The first oligomeric procyanolidins used as component (b) were isolated from grape seeds by Masquellier. They contain the tannins widespread in plants as monomer units. Chemically, there are two types of tannins, namely condensed forms, including the procyanidin A2, and hydrolyzed tannins. Condensed tannins—also known as flavolans—are formed in the biosynthesis by condensation of monomers such as, for example, catechin, gallocatechin, afzelechin (2-R, 3-S type monomers) and epicatechin, epigallocatechin and epiafzelechin (2-R, 3-R type monomers). First dimers and then higher oligomers are formed by condensation of the monomers, the condensation taking place by formation of a C—C bond in the 4-8 or 6-8 position. In the case of the preferred A2 dimers of the proanthocyanidin A2 type, there is a double bond, namely C2>0>C7 and C4>C8. The structure is shown below:

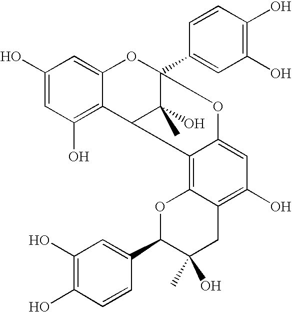

The A2 type proanthocyanidins are less susceptible to hydrolysis than the B types. Also, this term is used synonymously for the group of condensed tannins because—they eliminate monomers under the influence of hot mineral acids. Basically, the proanthocyanidins may be synthetic although enrichment products containing an effective quantity of OPC or A2 dimers, which may be obtained by extraction of certain fruits, seeds, plants or plant parts, are preferred from the practical perspective. Sources include, in particular, green tea (Camellia sinensis), pine bark (Pinia silvestris), grape seed (Vitis vinifera), Litchi pericarp (Litchi sinensis) and potentilla (Potentille erecta) and mixtures thereof.

Other suitable additives are the caffeine-containing and astringent or diuretic extracts of Guarana and Java tea.

Extraction

The proanthocyanilidone-containing extracts may be prepared by methods known per se, i.e. for example by aqueous, alcoholic or aqueous/alcoholic extraction of the plants or parts thereof or the leaves or fruit. Suitable extraction processes are any of the usual extraction processes such as, for example, maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux. Percolation is advantageous for industrial use. Fresh plants or parts thereof are suitable as the starting material although dried plants and/or plant parts which may be mechanically size-reduced before extraction are normally used. Any size reduction methods known to the expert, for example freeze grinding, may be used. Preferred solvents for the extraction process are organic solvents, water (preferably hot water with a temperature above 80° C. and more particularly above 95° C.) or mixtures of organic solvents and water, more particularly low molecular weight alcohols with more or less high water contents. Extraction with methanol, ethanol, pentane, hexane, heptane, acetone, propylene glycols, polyethylene glycols, ethyl acetate and mixtures and water-containing mixtures thereof thereof is particularly preferred. The extraction process is generally carried out at 20 to 100° C., preferably at 30 to 90° C. and more particularly at 60 to 80° C. In one preferred embodiment, the extraction process is carried out in an inert gas atmosphere to avoid oxidation of the ingredients of the extract. This is particularly important where extraction is carried out at temperatures above 40° C. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried leaves are in the range from 3 to 15 and more particularly 6 to 10% by weight. The present invention includes the observation that the extraction conditions and the yields of the final extracts may be selected according to the desired application. These extracts, which generally have active substance contents (=solids contents) of 0.5 to 10% by weight, may be used as such, although the solvent may also be completely removed by drying, more particularly by spray or freeze drying. The extracts may also be used as starting materials for producing the pure active substances mentioned above unless they can be synthesized by a more simple and inexpensive method. Accordingly, the active substance content in the extracts may be from 5 to 100% by weight and is preferably from 50 to 95% by weight. The extracts themselves may be present as water-containing preparations and/or as preparations dissolved in organic solvents and as spray-dried or freeze-dried water-free solids. Suitable organic solvents in this connection are, for example, aliphatic alcohols containing 1 to 6 carbon atoms (for example ethanol), ketones (for example acetone), halogenated hydrocarbons (for example chloroform or methylene chloride), lower esters or polyols (for example glycerol or glycols).

Components (a) and (b) are preferably used in a ratio by weight of 90:10 to 10:90, special synergistic effects being observed in the range from 75:25 to 25:75 and especially in the range from 60:40 to 40:60.

Encapsulation

In one particular embodiment of the present invention, the oral preparations are used in encapsulated form—for example in the form of typical gelatin macrocapsules—but preferably in microencapsulated form. A typical gelatin capsule may contain, for example, 3 g CLA and 150 mg OPC for daily oral application.

“Microcapsules” are understood by the expert to be spherical aggregates with a diameter of about 0.0001 to about 5 mm which contain at least one solid or liquid core surrounded by at least one continuous membrane. More precisely, they are finely dispersed liquid or solid phases coated with film-forming polymers, in the production of which the polymers are deposited onto the material to be encapsulated after emulsification and coacervation or interfacial polymerization. In another process, molten waxes are absorbed in a matrix (“microsponge”) which, as microparticles, may be additionally coated with film-forming polymers. The microscopically small capsules, also known as nanocapsules, can be dried in the same way as powders. Besides single-core microcapsules, there are also multiple-core aggregates, also known as microspheres, which contain two or more cores distributed in the continuous membrane material. In addition, single-core or multiple-core microcapsules may be surrounded by an additional second, third etc. membrane. The membrane may consist of natural, semisynthetic or synthetic materials. Natural membrane materials are, for example, gum arabic, agar agar, agarose, maltodextrins, alginic acid and salts thereof, for example sodium or calcium alginate, fats and fatty acids, cetyl alcohol, collagen, chitosan, lecithins, gelatin, albumin, shellac, polysaccharides, such as starch or dextran, polypeptides, protein hydrolyzates, sucrose and waxes. Semisynthetic membrane materials are inter alia chemically modified celluloses, more particularly cellulose esters and ethers, for example cellulose acetate, ethyl cellulose, hydroxypropyl cellulose, hydroxypropyl methyl cellulose and carboxymethyl cellulose, and starch derivatives, more particularly starch ethers and esters. Synthetic membrane materials are, for example, polymers, such as polyacrylates, polyamides, polyvinyl alcohol or polyvinyl pyrrolidone.

Examples of known microcapsules are the following commercial products (the membrane material is shown in brackets) Hallcrest Microcapsules (gelatin, gum arabic), Coletica Thalaspheres (maritime collagen), Lipotec Millicapseln (alginic acid, agar agar), Induchem Unispheres (lactose, microcrystalline cellulose, hydroxypropylmethyl cellulose), Unicerin C30 (lactose, microcrystalline cellulose, hydroxypropylmethyl cellulose), Kobo Glycospheres (modified starch, fatty acid esters, phospholipids), Softspheres (modified agar agar), Kuhs Probiol Nanospheres (phospholipids), Primaspheres and Primasponges (chitosan, alginates) and Primasys (phospholipids).

Chitosan microcapsules and processes for their production are the subject of earlier patent applications filed by applicants [WO 01/01926, WO 01/01927, WO 01/01928, WO 01/01929]. Microcapsules with mean diameters of 0.0001 to 5, preferably 0.001 to 0.5 and more particularly 0.005 to 0.1 mm, which consist of a membrane and a matrix containing the active components, may be obtained, for example, by
(a1) preparing a matrix from gel formers, chitosans and active components,
(a2) optionally dispersing the matrix in an oil phase and
(a3) treating the dispersed matrix with aqueous solutions of anionic polymers and optionally removing the oil phase in the process or
(b1) preparing a matrix from gel formers, anionic polymers and active components,
(b2) optionally dispersing the matrix in an oil phase and
(b3) treating the dispersed matrix with aqueous chitosan solutions and optionally removing the oil phase in the process or
(c1) processing aqueous active-component preparations with oil components in the presence of emulsifiers to form o/w emulsions,
(c2) treating the emulsions thus obtained with aqueous solutions of anionic polymers,
(c3) contacting the matrix thus obtained with aqueous chitosan solutions and
(c4) removing the encapsulated products thus obtained from the aqueous phase.
   Gel Formers

Preferred gel formers for the purposes of the invention are substances which are capable of forming gels in aqueous solution at temperatures above 40° C. Typical examples of such gel formers are heteropolysaccharides and proteins. Preferred thermogelling heteropolysaccharides are agaroses which may be present in the form of the agar agar obtainable from red algae, even together with up to 30% by weight of non-gel-forming agaropectins. The principal constituent of agaroses are linear polysaccharides of D-galactose and 3,6-anhydro-L-galactose with alternate β-1,3- and β-1,4-glycosidic bonds. The heteropolysaccharides preferably have a molecular weight of 110,000 to 160,000 and are both odorless and tasteless. Suitable alternatives are pectins, xanthans (including xanthan gum) and mixtures thereof. Other preferred types are those which—in 1% by weight aqueous solution—still form gels that do not melt below 80° C. and solidify again above 40° C. Examples from the group of thermogelling proteins are the various gelatins.

Chitosans

Chitosans are biopolymers which belong to the group of hydrocolloids. Chemically, they are partly deacetylated chitins differing in their molecular weights which contain the following—idealized—monomer unit: 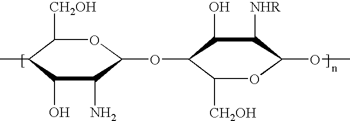
In contrast to most hydrocolloids, which are negatively charged at biological pH values, chitosans are cationic biopolymers under these conditions. The positively charged chitosans are capable of interacting with oppositely charged surfaces and are therefore used in cosmetic hair-care and body-care products and pharmaceutical preparations. Chitosans are produced from chitin, preferably from the shell residues of crustaceans which are available in large quantities as inexpensive raw materials. In a process described for the first time by Hackmann et al., the chitin is normally first deproteinized by addition of bases, demineralized by addition of mineral acids and, finally, deacetylated by addition of strong bases, the molecular weights being distributed over a broad spectrum. Preferred types are those which have an average molecular weight of 10,000 to 500,000 dalton or 800,000 to 1,200,000 dalton and/or a Brookfield viscosity (1% by weight in glycolic acid) below 5,000 mPas, a degree of deacetylation of 80 to 88% and an ash content of less than 0.3% by weight. In the interests of better solubility in water, the chitosans are generally used in the form of their salts, preferably as glycolates.
Oil Phase

Before formation of the membrane, the matrix may optionally be dispersed in an oil phase. Oils suitable for this purpose are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-/di-/triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv®) TN), linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons such as, for example, squalane, squalene or dialkyl cyclohexanes.

Anionic Polymers

The function of the anionic polymers is to form membranes with the chitosans. Preferred anionic polymers are salts of alginic acid. The alginic acid is a mixture of carboxyl-containing polysaccharides with the following idealized monomer unit:

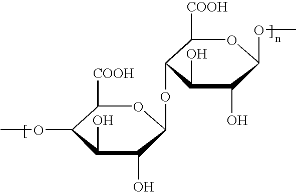

The average molecular weight of the alginic acid or the alginates is in the range from 150,000 to 250,000. Salts of alginic acid and complete and partial neutralization products thereof are understood in particular to be the alkali metal salts, preferably sodium alginate (“algin”), and the ammonium and alkaline earth metal salts. Mixed alginates, for example sodium/magnesium or sodium/calcium alginates, are particularly preferred. In an alternative embodiment of the invention, however, anionic chitosan derivatives, for example carboxylation and above all succinylation products are also suitable for this purpose. Alternatively, poly(meth)acrylates with average molecular weights of 5,000 to 50,000 dalton and the various carboxy-methyl celluloses may also be used. Instead of the anionic polymers, anionic surfactants or low molecular weight inorganic salts, such as pyrophosphates for example, may also be used for forming the membrane.

Production Process for Microcapsules

To produce the microcapsules, a 1 to 10 and preferably 2 to 5% by weight aqueous solution of the gel former, preferably agar agar, is normally prepared and heated under reflux. A second aqueous solution containing the chitosan in quantities of 0.1 to 2 and preferably 0.25 to 0.5% by weight and the active substances in quantities of 0.1 to 25 and preferably 0.25 to 10% by weight is added in the boiling heat, preferably at 80 to 100° C.; this mixture is called the matrix. Accordingly, the charging of the microcapsules with active substances may also comprise 0.1 to 25% by weight, based on the weight of the capsules. If desired, water-insoluble constituents, for example inorganic pigments, may be added at this stage to adjust viscosity, generally in the form of aqueous or aqueous/alcoholic dispersions. In addition, to emulsify or disperse the active substances, it can be useful to add emulsifiers and/or solubilizers to the matrix. After its preparation from gel former, chitosan and active substances, the matrix may optionally be very finely dispersed in an oil phase with intensive shearing in order to produce small particles in the subsequent encapsulation process. It has proved to be particularly advantageous in this regard to heat the matrix to temperatures in the range from 40 to 60° C. while the oil phase is cooled to 10 to 20° C. The actual encapsulation, i.e. formation of the membrane by contacting the chitosan in the matrix with the anionic polymers, takes place in the last, again compulsory step. To this end, it is advisable to wash the matrix optionally dispersed in the oil phase with an aqueous ca. 1 to 50 and preferably 10 to 15% by weight aqueous solution of the anionic polymer at a temperature of 40 to 100° C. and preferably at a temperature of 50 to 60° C. and, if necessary, to remove the oil phase either at the same time or afterwards. The resulting aqueous preparations generally have a microcapsule content of 1 to 10% by weight. In some cases, it can be of advantage for the solution of the polymers to contain other ingredients, for example emulsifiers or preservatives. After filtration, microcapsules with a mean diameter of preferably about 1 mm are obtained. It is advisable to sieve the capsules to ensure a uniform size distribution. The microcapsules thus obtained may have any shape within production-related limits, but are preferably substantially spherical. Alternatively, the anionic polymers may also be used for the preparation of the matrix and encapsulation may be carried out with the chitosans.

An alternative process for the production of the microcapsules according to the invention comprises initially preparing an o/w emulsion which, besides the oil component, water and the active components, contains an effective quantity of emulsifier. To form the matrix, a suitable quantity of an aqueous anionic polymer solution is added to this preparation with vigorous stirring. The membrane is formed by addition of the chitosan solution. The entire process preferably takes place at a mildly acidic pH of 3 to 4. If necessary, the pH is adjusted by addition of mineral acid. After formation of the membrane, the pH is increased to a value of 5 to 6, for example by addition of triethanolamine or another base. This results in an increase in viscosity which can be supported by addition of other thickeners such as, for example, polysaccharides, more particularly xanthan gum, guar guar, agar agar, alginates and tyloses, carboxymethyl cellulose and hydroxyethyl cellulose, relatively high molecular weight polyethylene glycol mono- and diesters of fatty acids, polyacrylates, polyacrylamides and the like. Finally, the microcapsules are separated from the aqueous phase, for example by decantation, filtration or centrifuging.

Commercial Applications

The preparations according to the invention taken orally show a synergistically improved inhibition of the lipogenase activity and the drainage function in the skin. Accordingly, the present invention also relates to the use of mixtures containing
(a) physiologically active fatty acids containing 16 to 26 carbon atoms and 2 to 6 double bonds, esters or glycerides thereof and
(b) oligomeric proanthocyanolidins (OPCs) or plant extracts containing them
   for the production of food additives, more especially for reducing body fat in the human or animal organism and for regulating the moisture content of the skin.

### EXAMPLES

### Example 1

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g conjugated linoleic acid (Tonalin® CLA-80), 1 g dried Vitis vinifera extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 2

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g of a technical omega-3 fish fatty acid mixture (Omacor®), 1 g dried Vitis vinifera extract K, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 3

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g CLA triglyceride (Tonalin® CLA-TG), 1 g dried pine bark extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 4

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g conjugated linoleic acid (Tonalin® CLA-80), 1 g dried Lichi chinensis extract, 0.5 g Phenonip®) and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 5

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g conjugated linoleic acid (Tonalin® CLA-80), 1 g dried Camellia sinensis extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

1-11. (canceled)

12. A composition for oral administration comprising:
(a) a physiologically active fatty acid containing about 16 to 26 carbon atoms and about 2 to 6 double bonds, and esters or glycerides thereof; and
(b) an oligomeric proanthocyanidin or a plant extract containing said proanthocyanidin.

13. The composition of claim 12, wherein component (a) is a conjugated linoleic acid and esters or glycerides thereof.

14. The composition of claim 13, wherein the conjugated linoleic acid contains at least 30% by weight t10,c12 isomers, at least 30% by weight c9,t11 isomers and less than 1% by weight 8,10-, 11,13- and t,t-isomers.

15. The composition of claim 12 wherein component (a) is an omega-3 fatty acid.

16. The composition of claim 12 wherein the oligomeric proanthocyanidin is of the A2 type of proanthocyanidin.

17. The composition of claim 12 wherein the plant extract of component (b) is selected from the group of plants consisting of Camellia sinensis, Pinia silvestris, Vitis vinifera, Litchi chinensis, Potentille erecta and mixtures thereof.

18. The composition of claim 12 wherein components (a) and (b) are present in a ratio by weight of from about 90:10 to 10:90.

19. The composition of claim 18 wherein components (a) and (b) are present in a ratio by weight of from about 60:40 to 40:60.

20. The composition of claim 12 which is provided in an encapsulated form.

21. The composition of claim 20 which is provided in a microencapsulated form.

22. A method of using a composition for oral administration, which method comprises incorporating into a food product said composition as an additive, which composition comprises:
(a) a physiologically active fatty acid containing about 16 to 26 carbon atoms and about 2 to 6 double bonds, and esters or glycerides thereof; and
(b) an oligomeric proanthocyanidin or a plant extract containing said proanthocyanidin.

23. The method of claim 22 wherein the food product is for reducing body fat in a mammal.

24. The method of claim 22 wherein the food product is for regulating the moisture content of skin.
